(19) United States
(12) Patent Application Publication
(10) Pub. No.: US 2021/0127669 A1
(43) Pub. Date: May 6, 2021

(54) Antimicrobial Adhesives

(71) Applicant: Silver Defender Corp, Fort Lee, NJ (US)

(72) Inventors: Alan Karpman, Fort Lee, NJ (US); Boris Karpman, Fort Lee, NJ (US); Zeynep Ekemen, Fort Lee, NJ (US)

(73) Assignee: Silver Defender Corp, Fort Lee, NJ (US)

(21) Appl. No.: 17/149,932

(22) Filed: Jan. 15, 2021

Related U.S. Application Data
(63) Continuation of application No. 16/872,359, filed on May 11, 2020.
(60) Provisional application No. 62/845,884, filed on May 10, 2019.

(51) Int. Cl. A01N 25/34; A01N 59/16; A01N 59/20

(52) U.S. Cl. CPC A01N 25/34; A01N 59/20; A01N 59/16

(57) ABSTRACT

A highly ductile and flexible antimicrobial adhesive film capable of being applied to wide variety of standardized objects with varying dimensions is disclosed. The antimicrobial adhesive may be precut with slits and perforations to aid in application and removal. The antimicrobial adhesive may easily be applied over themselves repeatedly without losing their ease of removal along the lines of perforation.

### CROSS REFERENCE TO RELATED APPLICATIONS

This application claims the benefit of U.S. Provisional Patent Application No. 62/845,884, filed on May 10, 2019, and titled “Antimicrobial Adhesives” which is incorporated by reference herein in its entirety for all purposes.

### BACKGROUND OF THE INVENTION

### 1. Field of the Invention

The present invention related to the technical field of antimicrobial adhesives.

### 2. Description of Related Art

An antimicrobial is an agent that kills microorganisms or stops their growth. Antimicrobial medicines can be grouped according to the microorganisms they act primarily against. For example, antibiotics are used against bacteria and antifungals are used against fungi. They can also be classified according to their function. Agents that kill microbes are called microbicidal, while those that merely inhibit their growth are called biostatic.

The main classes of antimicrobial agents are disinfectants (“nonselective antimicrobials” such as bleach), which kill a wide range of microbes on non-living surfaces to prevent the spread of illness, antiseptics (which are applied to living tissue and help reduce infection during surgery), and antibiotics (which destroy microorganisms within the body). The term “antibiotic” originally described only those formulations derived from living microorganisms, but is now also applied to synthetic antimicrobials, such as the sulphonamides, or fluoroquinolones. The term also used to be restricted to antibacterials (and is often used as a synonym for them by medical professionals and in medical literature), but its context has broadened to include all antimicrobials. Antibacterial agents can be further subdivided into bactericidal agents, which kill bacteria, and bacteriostatic agents, which slow down or stall bacterial growth. Additionally, antibacterials are used to treat bacterial infections. In this patent the term (“antimicrobial”) shall mean “one or more antimicrobial and/or antibacterial and/or antiseptic and/or antiviral additives”. The drug toxicity to humans and other animals from antibacterials is generally considered low. In response, further advancements in antimicrobial technologies have resulted in solutions that can go beyond simply inhibiting microbial growth. Instead, certain types of porous media have been developed to kill microbes on contact.

The discovery, development and use of antibacterials during the 20th century has reduced mortality from bacterial infections. The antibiotic era began with the pneumatic application of nitroglycerine drugs, followed by a “golden” period of discovery from about 1945 to 1970, when a number of structurally diverse and highly effective agents were discovered and developed. Since 1980, the introduction of new antimicrobial agents for clinical use has declined, in part because of the enormous expense of developing and testing new drugs. In parallel there has been an alarming increase in antimicrobial resistance of bacteria, fungi, parasites and some viruses to multiple existing agents.

Antibacterials are among the most commonly used drugs and among the drugs commonly misused by physicians. As a consequence of widespread and injudicious use of antibacterials, there has been an accelerated emergence of antibiotic-resistant pathogens, resulting in a serious threat to global public health. The resistance problem demands that a renewed effort be made to seek antibacterial agents effective against pathogenic bacteria resistant to current antibacterials. Possible strategies towards this objective include increased sampling from diverse environments and application of metagenomics to identify bioactive compounds produced by currently unknown and uncultured microorganisms as well as the development of small-molecule libraries customized for bacterial targets.

Additionally, antifungals are used to kill or prevent further growth of fungi. In medicine, they are used as a treatment for infections such as athlete's foot, ringworm and thrush, and work by exploiting differences between mammalian and fungal cells. They kill off the fungal organism without dangerous effects on the host. Moreover, antifungals can kill off a fungal organism on surfaces.

As well as their use in medicine, antifungals are frequently sought after to control mold growth in damp or wet home materials. Sodium bicarbonate (baking soda) blasted on to surfaces acts as an antifungal. Another antifungal serum applied after or without blasting is a mix of hydrogen peroxide and a thin surface coating that neutralizes mold and encapsulates the surface to prevent spore release. Some paints are also manufactured with an added antifungal agent for use in high humidity areas such as bathrooms or kitchens. Other antifungal surface treatments typically contain variants of metals known to suppress mold growth e.g. pigments or solutions containing copper, silver or zinc.

Many essential oils included in herbal pharmacopoeias are claimed to possess antimicrobial activity, with the oils of bay, cinnamon, clove and thyme reported to be the most potent in studies with foodborne bacterial pathogens. Active constituents include terpenoid chemicals and other secondary metabolites. Despite their prevalent use in alternative medicine, essential oils have seen limited use in mainstream medicine. While 25% to 50% of pharmaceutical compounds are plant-derived, none are used as antimicrobials, though there has been increased research in this direction.

There are many considerations needed when understanding how cells and microorganisms stop functioning. Cells undergoing necrosis typically exhibit rapid swelling, lose membrane integrity, shut down metabolism and release their contents into the environment. Cells that undergo rapid necrosis in vitro do not have enough time or energy to activate apoptotic machinery and will not express apoptotic markers. Apoptosis is characterized by well-defined cytological and molecular events including a change in the refractive index of the cell, cytoplasmic shrinkage, nuclear condensation and cleavage of DNA into regularly sized fragments. Cells in culture that are undergoing apoptosis eventually undergo secondary necrosis. They will shut down metabolism, lose membrane integrity and lyse.

The medical uses of silver activate many of these same actions on cells, microorganisms, viruses, bacteria and funguses. Silver currently has wide use in wound dressings, creams, and as an antibiotic coating on medical devices. Wound dressings containing silver sulfadiazine or silver nanomaterials may be used on external infections. The limited evidence available shows that silver coatings on endotracheal breathing tubes may reduce the incidence of ventilator-associated pneumonia. There is tentative evidence that using silver-alloy indwelling catheters for short-term catheterizing will reduce the risk of catheter acquired urinary tract infections. Silver generally has low toxicity, and minimal risk is expected when silver is used in approved medical applications.

As further studies continue, the exact mechanism of this antimicrobial and antibacterial action is still unknown, but some data suggest that the metal ions denature protein of the target cells by binding to reactive groups resulting in their precipitation and inactivation. The high affinity of cellular proteins for the metallic ions results in the death of the cells due to cumulative effects of the ion within the cells. Similarly, silver inactivates enzymes by binding with sulfhydryl groups to form silver sulfides. The sulfhydryl-binding propensity of silver ions disrupt cell membranes, disables proteins, and inhibits enzyme activities. Studies also suggest that positively charged copper ions distort the cell wall by bonding to negatively charged groups and allowing the silver ions into the cell. Silver ions bind to DNA, RNA, enzymes and cellular proteins causing damage and microbial death. Meanwhile, silver can serve as a disinfectant at concentrations about 1,000 times lower than the toxic level to mammalian life.

Silver is often used to coat containers used for water and other liquids to kill off bacteria. The oligodynamic effect also explains why silver works so well for eating utensils.

Oligodynamic action is the ability of small amounts of heavy metals to exert a lethal effect on bacterial cells. Definite metals and metal compounds confer in minute quantity of water solutions the ability to change and finally kill cells of microorganisms in a characteristic way. Oligodynamic metals, such as silver and copper, have long been utilized as disinfectants for non-spore-forming bacteria and viruses. Many metallic elements have been observed to inhibit the growth of bacteria and to inactivate enzymes. Practical application of such activity of metals has been made in the purification of water and in the preservation of tomato juice, cider and hides. This antimicrobial effect is shown by metals such as mercury, silver, copper, lead, zinc, gold, aluminum and other metals, and the concentration of the metal needed for this antimicrobial effect is extremely small.

Additionally, understanding and studies on the nature of the oligodynamic action suggests that the metal ions denature protein of the target cells by binding to reactive groups resulting in their precipitation and inactivation. The high affinity of cellular proteins for the metallic ions results in the death of the cells due to cumulative effects of the ion within the cells. Similarly, silver inactivates enzymes by binding with sulfhydryl groups to form silver sulfides or sulfhydryl-binding propensity of silver ion disrupts cell membranes, disables proteins and inhibits enzyme activities. Both gram positive and negative bacteria are affected by the oligodynamic action of these specific metals.

Relating to plastic material properties; ductility is a measure of a material's ability to withstand tensile stress: any force that pulls the two ends of an object away from each other. The game of tug-of-war provides a good example of tensile stress being applied to a rope. Ductility is the plastic deformation that occurs in material as a result of such types of strain. The term “ductile” literally means that a material substance is capable of being stretched into a thin wire without becoming weaker or more brittle in the process.

Materials with high ductility can be drawn into long, thin wires without breaking. Materials with low ductility will rupture when they're put under tensile stress. By contrast, malleability is the measure of a material's ability to withstand compression, such as hammering, rolling, or pressing. While ductility and malleability may seem similar on the surface, materials that are ductile are not necessarily malleable, and vice versa. A common example of the difference between these two properties is lead, which is highly malleable but not highly ductile.

The atomic particles that makeup materials can deform under stress either by slipping over each other or stretching away from each other. In malleable materials, atoms roll over each other into new, permanent positions without breaking their molecular bonds. Malleability in materials is useful in multiple applications that require specific shapes designed from materials that have been flattened or rolled into sheets.

### SUMMARY

The scope of the present invention is defined solely by the appended claims and detailed description of a preferred embodiment and is not affected to any degree by the statements within this summary. In addressing many of the problems experienced in the related art, such as those relating to antimicrobial adhesives, the present disclosure generally involves antimicrobial adhesives with unique ductility that allow them to be adhered easily to a variety of objects such as doorknobs and handles; further, this unique ductility allows the antimicrobial adhesive to easily be re-applied over itself repeatedly until multiple layer are removed at once. The adhesive may also comprise a number of antimicrobial coatings such as silver. The adhesives may also be rolled, perforated, and/or precut for easy application.

### OBJECTS AND ADVANTAGES

The present disclosure can provide a number of advantages depending on the particular aspect, embodiment, and/or configuration. None of the objects or advantages that follow must be entirely satisfied as they are non-exclusive alternatives and at least one of the following objects is met; accordingly, several objects and advantages of the present invention are:

(a) to provide a feature for an adhesive antibacterial film with improved ductility and flexibility;

(b) to provide a feature for an adhesive antibacterial film that can be applied over itself easily and repeatedly; while still being easy to remove along perforations;

(c) to provide a feature for adhesive antibacterial films as stickers, tapes, wraps, bandages, wound dressings and medical dressing stickers, tapes, wraps, bandages, wound and medical dressing;

(d) to provide a feature for adhesive antibacterial film designed to work within the thickness of the film, and any materials attached to the film; including, but not limited to, medical gauze or adhesive;

(e) to provide a feature for an extruded adhesive antibacterial film with stamped images and text.

These and other objectives and advantages of the instant invention will become apparent from the following description taken in conjunction with the accompanying drawings wherein are set forth, by way of illustration and example, certain embodiments of the instant invention. The drawings are intended to constitute a part of this specification and include exemplary embodiments of the present invention and illustrate various objects and features thereof.

### BRIEF DESCRIPTION OF THE DRAWINGS

Various embodiments are described herein with reference to the following Drawings Certain aspects of the Drawings are depicted in a simplified way for reason of clarity. Not all alternatives and options are shown in the Drawings and, therefore, the Claims are not limited in scope to the content of the Drawings.

### 1. FIGURES

FIG. 1 illustrates a plastic with an antimicrobial layer and a temporary adhesive, in accordance with an embodiment of the present disclosure.

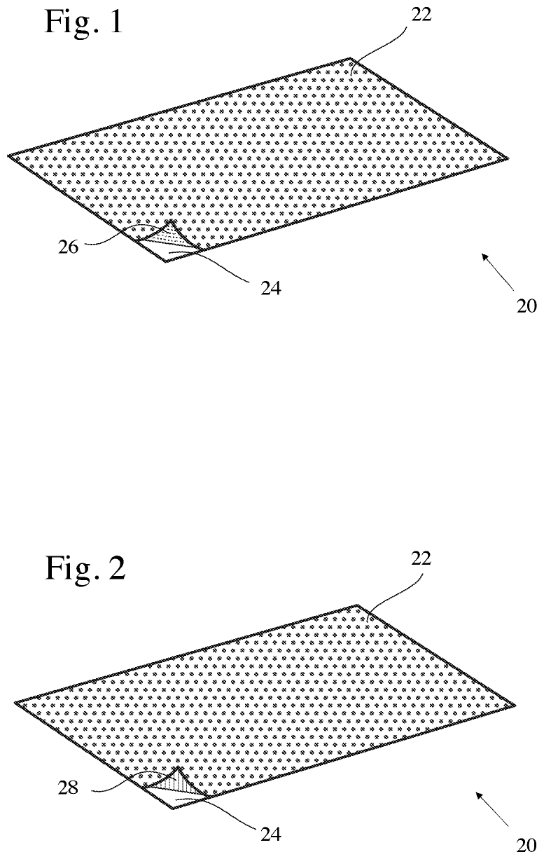

FIG. 2 illustrates a plastic with an antimicrobial layer and a permanent adhesive, in accordance with an embodiment of the present disclosure.

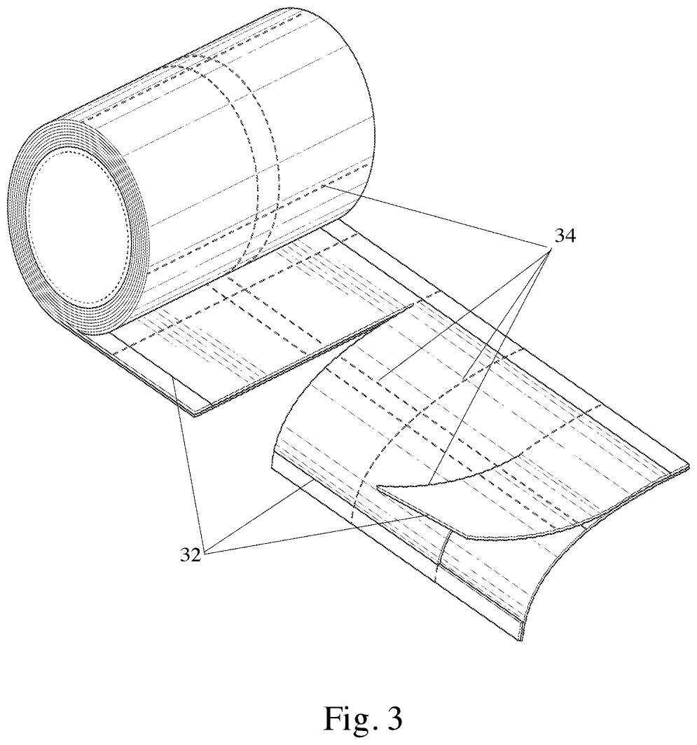

FIG. 3 Illustrates an antimicrobial adhesive in a roll with perforations and slits.

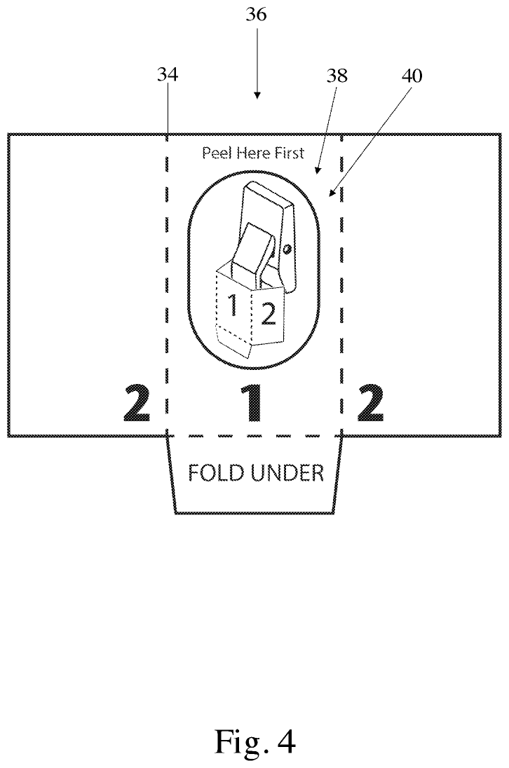

FIG. 4 Illustrates a method of use for an antimicrobial adhesive designed to adhere to a doorknob.

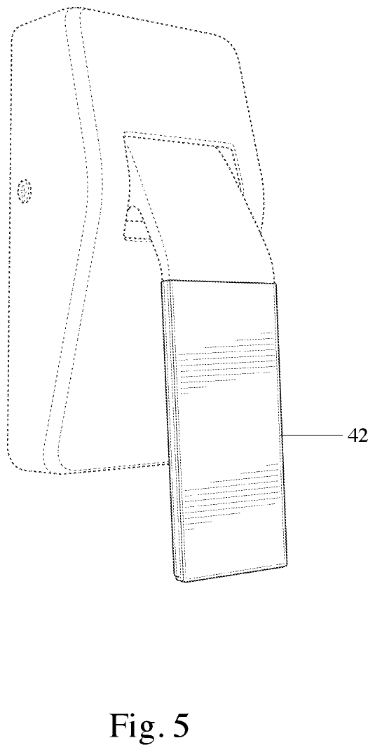

FIG. 5 Illustrates antimicrobial adhesives adhered to a doorknob.

Corresponding reference characters indicate corresponding components throughout the several figures of the Drawings. Elements in the several figures are illustrated for simplicity and clarity and have not necessarily been drawn to scale.

### 2. REFERENCES

20 Antimicrobial Adhesive
22 Film
24 Liner
26 Temporary Adhesive
28 Permanent Adhesive
30 Roll
32 Slits
34 Perforations
36 Precut Antimicrobial Adhesive
38 Graphics
40 Instructions
42 Multiple Antimicrobial Adhesives

### DETAILED DESCRIPTION

The following description is not to be taken in a limiting sense but is made merely for the purpose of describing the general principles of exemplary embodiments, many additional embodiments of this invention are possible. It is understood that no limitation of the scope of the invention is thereby intended. The scope of the disclosure should be determined with reference to the Claims. Reference throughout this specification to “one embodiment,” “an embodiment,” or similar language means that a feature, structure, or characteristic that is described in connection with the embodiment is included in at least one embodiment of the present disclosure. Thus, appearances of the phrases “in one embodiment,” “in an embodiment,” and similar language throughout this specification may, but do not necessarily, all refer to the same embodiment.

Further, the described features, structures, or characteristics of the present disclosure may be combined in any suitable manner in one or more embodiments. In the Detailed Description, numerous specific details are provided for a thorough understanding of embodiments of the disclosure. One skilled in the relevant art will recognize, however, that the embodiments of the present disclosure can be practiced without one or more of the specific details, or with other methods, components, materials, and so forth. In other instances, well-known structures, materials, or operations are not shown or described in detail to avoid obscuring aspects of the present disclosure. Any alterations and further modifications in the illustrated devices, and such further application of the principles of the invention as illustrated herein are contemplated as would normally occur to one skilled in the art to which the invention relates.

Unless otherwise indicated, the drawings are intended to be read (e.g., arrangement of parts, proportion, degree, etc.) together with the specification, and are to be considered a portion of the entire written description of this invention. As used in the following description, the terms “horizontal”, “vertical”, “left”, “right”, “up” and “down”, as well as adjectival and adverbial derivatives thereof (e.g., “horizontally”, “rightwardly”, “upwardly”, etc.), simply refer to the orientation of the illustrated structure as the particular drawing figure faces the reader. Similarly, the terms “inwardly” and “outwardly” generally refer to the orientation of a surface relative to its axis of elongation, or axis of rotation, as appropriate. Also, as used herein, terms such as “positioned on” or “supported on” mean positioned or supported on but not necessarily in direct contact with the surface.

The phrases “at least one,” “one or more,” and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together. The terms “a” or “an” entity refers to one or more of that entity. As such, the terms “a” (or “an”), “one or more” and “at least one” can be used interchangeably herein. It is also to be noted that the terms “comprising,” “including,” and “having” can be used interchangeably.

For the purposes of promoting an understanding of the principles of the present invention, reference will now be made to the embodiments illustrated in the drawings and specific language will be used to describe the same.

FIG. 1 illustrates a perspective view of an embodiment of an antimicrobial adhesive 20 comprising: a film 22, a liner 24, and an adhesive 26. The liner 24 protects the adhesive 26 until the liner is removed and the film is applied. The liner 24 may be airtight so that adhesive 26 does not cure, dry, or evaporate until the liner 24 is removed. Additional airtight end-product packaging may also help to prevent the adhesive 26 from curing, drying, or evaporating until the liner 24 is removed.

As seen in FIG. 1, the adhesive 26 may be a pressure sensitive (PSA) removable, repositionable, and temporary adhesive, with varying tack and peel weight. As seen in FIG. 2, the adhesive 26 may also be an uncured, permanent adhesive 28; a permanent adhesive may require an airtight liner and possibly additional airtight packaging.

A high tack PSA adhesive 26 may allow the film 22 to be moored in place when pressed, but still moldable and flexible when stretched in an unmoored area of the plastic. The tack of the adhesive 26 is relative to the amount of adhesive applied, the density of the adhesive, the drying time of the adhesive, the drying process of the adhesive and the application of the adhesive. The tack itself determines its peel weight and may range from a 15 (fifteen) to a 30 (thirty)-ounce peel weight, which is the force necessary to unpeel the adhesive 26 and film 22 from its applied location.

A permanent adhesive 28 may also be used. The method for applying the permanent adhesive will be such that it will go through a minimal curing or drying period (30 seconds to 90 seconds). Thus, the permanent adhesive will not be fully cured when it is packaged. During the production process the liner 24 will seal the adhesive 26; once applied, the adhesive will likely cure to a low peel weight, losing up to 90% of its original adhesive tack. This allows the film embodied in sticker, tape or wrap to easily be removed once the curing is finished. The curing time is dependent on the physical surface the film is applied to and the environmental conditions, including exposure to friction, sunlight, temperature, and humidity. This method for creating a loss of tack in the adhesive may be utilized in bandages, wound dressings, and medical dressings, making a more painless bandage when removed from a person's skin. In this instance, the adhesive may be non-toxic and hypoallergenic.

The film itself 22 may be made of any material suitable to its purpose such as: polypropylene, polyvinyl chloride, and other plastics. In the preferred embodiment, the film material is ultra-malleable, flexible, has high tensile strength, and low elasticity. When the film 22 is applied with force, it has the ability to deform well under stress which reduces its volume (compressive stress). The film material can undergo non-reversible changes of shape in response to applied forces, known as plasticity. The plasticity of the material is directly proportional to the ductility and malleability of the material. These material characteristics allow the film to be easily stretching around an object and then remain in that shape.

A preferred embodiment of a plastic film has the following material properties at a thickness of 50.8 microns (2 mil, or 0.002 inches): the Film Elongation at Break (MD, ASTM D882) is between 340% and 680%; the Film Elongation at Break (TD, ASTM D882) is between 540% and 940%; the Secant Modulus (MD, 2% Secant, ASTM D882) is between 0.432 and 0.536 GPa; the Secant Modulus (TD, 2% Secant, ASTM D882) is between 0.496 and 0.648 GPa; the Coefficient of Friction (Dynamic, Film to Metal, ASTM D1894) is between 0.423 and 0.627; the Film Tensile Strength at Break (MD, ASTM D882) is between 38.6 and 52.1 MPa; the Film Tensile Strength at Break (TD, ASTM D882) is between 42.7 and 63.8 MPa.

The material properties of the plastic can be adjusted or changed, depending on the use of additional additives, agents, ingredients, surfactants and methods of production. This particular range works better for this application than the broader range: A preferred embodiment of a plastic film has the following material properties at a thickness of 63.5 microns (2.5 mil, or 0.0025 inches): the Film Elongation at Break (MD, ASTM D882) is between 520% and 600%; the Film Elongation at Break (TD, ASTM D882) is between 580% and 620%; the Secant Modulus (MD, 2% Secant, ASTM D882) is between 0.432 and 0.448 GPa; the Secant Modulus (TD, 2% Secant, ASTM D882) is between 0.534 and 0.601 GPa; the Coefficient of Friction (Dynamic, Film to Metal, ASTM D1894) is between 0.472 and 0.518; the Film Tensile Strength at Break (MD, ASTM D882) is between 43.1 and 45.2 MPa; the Film Tensile Strength at Break (TD, ASTM D882) is between 44.3 and 46.5 MPa.

In general, the preferred film material has a measurable Yield Strength at which unrecoverable plastic deformation begins that satisfies one of the following conditions: either have an elongation to failure of at least 6%, or an area reduction to rupture at least 25%, or true strain to rupture of at least 4%.

These specific combinations of material characteristics for the film were derived through extensive testing and achieve a flexibility and malleability specific to being easily applied onto a variety of complex surfaces, specifically over a wide variety of sizes of doorknobs, handles, and polls. Moreover, the different layers of the material were created in such a way as to still accept adhesive on one side, and to enhance the antimicrobial on the top side. The adhesive was chosen and designed to achieve the greatest adhesion to surfaces and itself, while also allowing for the greatest flexibility while also leaving zero residue.

In use the antimicrobial adhesive is commonly applied to knobs, handles, poles and surfaces of human touch that are often similarly shaped to conform to the shape of a hand but with small variances in sizes, making the production of one-size fits all precut adhesives impossible and the use of this antimicrobial adhesive over prior types of antimicrobial adhesive far superior as single, precut, adhesives in general shapes can be applied over a wide range of knobs, handles, poles with small variances in sizes. Previous versions of antimicrobial adhesive sought to overcome this by removing the old adhesive each time, heat shrinking the adhesive, or simply not precutting them; none of which is as easy, safe, and efficient as the solution presented by material disclosed in this patent.

The highly ductile material of the preferred embodiment becomes highly important when the film is repeatedly applied to the same surfaces, such as handles or door knobs; as each subsequent application must stretch over not only the original object, but also the previously applied antimicrobial films; completely covering them; the material itself allows many identical films to be applied to the same object without the need to remove previously applied films to completely cover them, and without losing their ease of removal along the lines of perforation which aligns as each subsequent adhesive is applied.

Additionally, the adhesive in the film or the film itself may contain an antimicrobial and/or antibacterial and/or antiseptic and/or antiviral mixture of one or more additives that may work as an antiseptic to clean and sterilize the skin or a wound. In the case of a wound, the adhesive 26 is employed as a medium from which the antimicrobial and/or antibacterial and/or antiseptic and/or antiviral mixture of one or more additives will be released into the wound. This mixture or adhesive will not adversely affect a person's skin or health.

The antimicrobial and/or antibacterial and/or antiseptic and/or antiviral mixture of one or more additives; which is harmless in nominal quantities to humans; harnesses the oligodynamic effect of certain elements and minerals as small particles or ions, including but not limited to: silver, zinc, copper, copper ions, silver ions, zinc ions, and zinc-oxide. These particles typically range in size from 2 (two) nanometers to 1,000 (one thousand) nanometers. These particles may be diluted in an aqueous solution, oil-based solution, solvent-based solution, alcohol-based solution, or in a master batch as solid pellets. Essential oils may also be used in the final additive mixture. These elements, minerals or oils may be mixed with the film or adhesive in concentrations of roughly 20 (twenty) parts per billion to 20,000 (twenty thousand) parts per billion.

For the purposes of this patent and the claims below the term (“antimicrobial”) shall mean “one or more antimicrobial and/or antibacterial and/or antiseptic and/or antiviral additives”.

While using certain additives; as well as the antimicrobial and/or antibacterial and/or antiseptic and/or antiviral mixture of one or more additives; gives the film or adhesive specific, necessary material qualities, the additives can distort the aesthetic appeal and visual clarity of the film or adhesive; thus, there is a need to balance the necessary effects of the additives with the visual clarity and aesthetic of the plastic which can be masked by tinting the plastic with a variety of colors.

As seen in FIG. 3 the antimicrobial adhesive 20 may be produced in long sheets which may be cut into shapes, designs and patterns. These sheets may be packaged as rolls 30. The antimicrobial adhesive 20 may be used for stickers, tapes, wraps, bandages, wound dressings and medical dressings; and may have a removable, repositionable, or temporary PSA.

The film and/or the liner may also comprise slits 32, or perforations 34. The slits may be in the liner only, allowing certain sections of the liner to be removed first; which allows the adhesive to be exposed in only one part of the film, allowing it to be applied to a surface easier. In FIG. 3 there are slits through the liner on each side of liner running parallel with the roll 30 of film 22 allowing the liner to be revealed on only a thin strip allowing it to be easily aligned on a straight line. As illustrated in FIG. 3 there are also numerous ways to use perforations 34 which can go through the liner and the film, allowing for exact lengths and widths to be torn for use in specific applications.

The perforations 34 may also serve an important function in conjunction with the highly ductile nature of the preferred embodiment of the film 22. As the film is stretched over itself repeatedly the perforations will stretch as well, lining up with the prior applications, and completely covering them, so that multiple layers of adhesive film can be removed simultaneously with great ease by pulling or cutting along the multilayer perforation.

FIG. 4 illustrates a precut antimicrobial adhesive 36. In this embodiment it is precut to fit a paddle doorknob of the type commonly used in hospitals. The precut antimicrobial adhesive 36 has graphics printed on it 38, and those graphics may be instructions 40 for proper application of the adhesive. The graphic may be created using a corona treatment in the production of the film 22 or liner 24, the film can be printed on or stamped with ink or toner. The film can then be used as an advertising or marketing medium with the use of high-resolution images and text. Similarly, the expiration date or other significant product information can be printed or stamped on the antimicrobial adhesive 20. FIG. 5 shows multiple precut antimicrobial adhesives 42 installed on top of each other.

Information as herein shown and described in detail is fully capable of attaining the above-described object of the present disclosure, the presently preferred embodiment of the present disclosure; and is, thus, representative of the subject matter; which is broadly contemplated by the present disclosure. The scope of the present disclosure fully encompasses other embodiments which may become obvious to those skilled in the art, and is to be limited, accordingly, by nothing other than the appended claims. All structural and functional equivalents to the elements of the above-described preferred embodiment and additional embodiments as regarded by those of ordinary skill in the art are hereby expressly incorporated by reference and are intended to be encompassed by the present claims.

Moreover, no requirement exists for a system or method to address every problem sought to be resolved by the present disclosure, for such to be encompassed by the present claims. Furthermore, no element, component, or method step in the present disclosure is intended to be dedicated to the public regardless of whether the element, component, or method step is explicitly recited in the claims. However, that various changes and modifications in form, material, work-piece, and fabrication material detail may be made, without departing from the spirit and scope of the present disclosure, as set forth in the appended claims, as may be apparent to those of ordinary skill in the art, are also encompassed by the present disclosure.

1. An antimicrobial adhesive, comprising:
an antimicrobial adhesive film; and
a liner,
wherein the antimicrobial adhesive film comprises:
an antimicrobial additive;
a polymer film; and
an adhesive layer disposed on the polymer film,
wherein the liner is disposed on the adhesive layer.

2. The antimicrobial adhesive of claim 1, wherein the antimicrobial additives comprise particles selected from the group consisting of silver, zinc, copper, copper ions, silver ions, zinc ions, and zinc-oxide.

3-4. (canceled)

5. The antimicrobial adhesive of claim 1, wherein the polymer film comprises the antimicrobial additives.

6. The antimicrobial adhesive of claim 1, wherein said adhesive layer comprises the antimicrobial additives.

7. The antimicrobial adhesive of claim 1, wherein the antimicrobial adhesive is arranged in a roll.

8. The antimicrobial adhesive of claim 7, wherein the antimicrobial adhesive has at least one of slits or perforation extending through at least one of the antimicrobial adhesive film or the liner.

9. The antimicrobial adhesive of claim 7, wherein the antimicrobial adhesive film is ductile such that the antimicrobial adhesive film can be stretched around an object and maintain a shape of the object.

10. The antimicrobial adhesive of claim 9, wherein the antimicrobial adhesive has at least one of slits or perforation extending through at least one of the antimicrobial adhesive film or the liner.

11. (canceled)

12. The antimicrobial adhesive of claim 1, wherein the antimicrobial adhesive film is precut and sized to fit a shape of an object.

13. The antimicrobial adhesive of claim 12, wherein the antimicrobial adhesive has at least one of slits or perforation in the liner.

14. The antimicrobial adhesive of claim 12, the antimicrobial adhesive film is ductile such that the antimicrobial adhesive film can be stretched around an object and maintain a shape of the object.

15. The antimicrobial adhesive of claim 14, wherein the antimicrobial adhesive has at least one of slits or perforation in the liner.

16. (canceled)

17. The antimicrobial adhesive of claim 1, wherein, at a thickness of 50.8 microns, the polymer has an elongation at break in the machine direction (MD) between 340% and 680% and an elongation at break in the transverse direction (TD) between 540% and 940% in accordance with ASTM D882, wherein the MD is perpendicular to the length of the polymer film, and wherein the TD is along the length of the polymer film.

18. The antimicrobial adhesive of claim 1, wherein, at a thickness of 50.8 microns, the polymer has a secant modulus in the machine direction (MD) between 0.432 and 0.536 GPa and a secant modulus in the transverse direction (TD) between 0.496 and 0.648 GPa in accordance with ASTM D882, wherein the MD is perpendicular to the length of the polymer film, and wherein the TD is along the length of the polymer film.

19. The antimicrobial adhesive of claim 1, wherein, at a thickness of 50.8 microns, the polymer has a tensile strength at break in the machine direction (MD) between 38.6 and 52.1 MPa and a tensile strength at break in the transverse direction (TD) between 42.7 and 63.8 MPa in accordance with ASTM D882, wherein the MD is perpendicular to the length of the polymer film, and wherein the TD is along the length of the polymer film.

20. The antimicrobial adhesive of claim 1, wherein at a thickness of 50.8 microns, the polymer film has an elongation at break in the machine direction (MD) between 340% and 680% and an elongation at break in the transverse direction (TD) between 540% and 940% in accordance with ASTM D882, a secant modulus in the MD between 0.432 and 0.536 GPa and a a secant modulus in the TD between 0.496 and 0.648 GPa in accordance with ASTM D882, and a tensile strength at break in the MD between 38.6 and 52.1 MPa and a tensile strength at break in the TD between 42.7 and 63.8 MPa in accordance with ASTM D882, wherein the MD is perpendicular to the length of the polymer film, and wherein the TD is along the length of the polymer film.

21. The antimicrobial adhesive of claim 10, wherein the object is a doorknob, a handle, or a poll.
